(19) United States
(12) Patent Application Publication
(10) Pub. No.: US 2025/0174313 A1
(43) Pub. Date: May 29, 2025

(54) METHODS AND SYSTEMS FOR ALCHEMICAL BINDING FREE ENERGY CALCULATIONS

(71) Applicants: Insilico Medicine IP Limited, Hong Kong (HK); Insilico Medicine Al Limited, Abu Dhabi (AE)

(72) Inventors: Vladimir Aleksandrovich Aladinskiy, Pak Shek Kok (HK); Georgy Andreev, Moscow (RU); Evgeny Kirilin, Nizhnekamsk (RU); Aleksandrs Zavoronkovs, Pak Shek Kok (HK)

(21) Appl. No.: 18/959,308

(22) Filed: Nov. 25, 2024

Related U.S. Application Data
(60) Provisional application No. 63/603,556, filed on Nov. 28, 2023.

(51) Int. Cl. G16C 20/50; G06F 30/27; G16C 10/00; G16C 20/70

(52) U.S. Cl. CPC G16C 20/50; G06F 30/27; G16C 10/00; G16C 20/70

(57) ABSTRACT

A computer-implemented method can include: obtaining a plurality of chemical compounds each having an initial condition and a final condition; computing comparative distances of areas of each of the plurality of chemical compounds in the initial condition and in the final condition, wherein the distances are calculated based on a physical configuration and electrical potential of each chemical compound; forming one or more clusters of the plurality of chemical compounds based on the computed comparative distances of the areas of each of the plurality of chemical compounds; and generating a diagram of changes for the plurality of chemical compounds, the diagram including a plurality of nodes and a plurality of edges, wherein each node indicates one chemical compound, and each edge is a transition from one chemical compound to another chemical compound of the plurality of chemical compounds; sampling conformational states of chemical compounds, preserving reliable microstates and ensuring an optimal transition pathway along with reproducible free energy estimates.

### CROSS-REFERENCE TO RELATED APPLICATIONS

This patent application claims priority to U.S. Provisional Application No. 63/603,556 filed Nov. 28, 2023, which provisional is incorporated herein by specific reference in its entirety.

### BACKGROUND

### Field

The present disclosure relates to systems and methods for calculating free energy differences and analyzing alchemical transformations. Particularly, the present disclosure relates to a method of generating networks of alchemical transformations and a protocol to perform such transformations.

### Description of Related Art

Alchemical calculations involve transforming a system from a first, or reference, thermodynamic state, denoted here as [symbol] , into a second, or target, thermodynamic state, denoted as [symbol] , along a pathway determined by a coupling parameter λ. The Helmholtz free energy ΔF of transformation from [symbol] to [symbol] may be defined for a pathway with M-2 intermediate thermodynamic states as a sum of contributions obtained using the free energy perturbation formula from each intermediate state along the path:

$$Δ {F}_{𝒜ℬ} = - \frac{1}{β} {\sum }_{i = 1}^{M - 1} ln {〈 {e}^{- βΔ {U}_{{λ}_{i} , {λ}_{i + 1}}} 〉}_{i}$$

$$where β = 1 / ( {k}_{B} T )$$

(k_{B} denotes the Boltzmann constant and T is the temperature), [symbol] . . . [symbol] represents an ensemble average over exp (−βU_{λ}), and ΔU_{λ}_{ᵢ}_{,λ}_{ᵢ₊₁}=U_{λ+1}−U_{λi} (U_{λ}_{ₖ}, is the potential energy of the system at the value of coupling parameter λₖ. In practical applications, alchemical calculations may be used to compute free energy differences associated with transfer processes, such as the binding of a small molecule to a protein receptor, the transfer of a chemical species between various physicochemical environments including but not limited to water or nonpolar solvents, mutual exchange of binding poses for a selected compound in order to prioritize them, the effects of protein side chain mutations on binding affinities or thermostabilities, among others.

A traditional pipeline of alchemical calculations may generally include system setup, transformation network (known as an alchemical network) generation, simulation setup, data analysis, and interpretation. The system setup may include defining a reference and a target (also known as a ghost) state which the system is set up to compare (e.g., calculate the free energy differences). Both reference and target states are also termed as endstates. In alchemical simulations, the Hamiltonian of the system is modified to interpolate between the reference and target states of the molecules involved. This interpolation is typically governed by a coupling parameter, commonly denoted as lambda (2), which varies smoothly from 0 to 1. At λ=0, the system represents the reference state (e.g., the unbound ligand), and at λ=1, it represents the target state (e.g., the bound ligand or a mutated form). Traditional methods for alchemical transformations often employ equilibrium techniques, such as thermodynamic integration (TI) or free energy perturbation (FEP), which require sampling at multiple intermediate lambda states. Nonequilibrium Switching (NEQS) techniques involve driving the system from the reference to the target state (or vice versa) in a finite amount of time, without the requirement of equilibrating at intermediate states. By performing multiple nonequilibrium simulations in both forward and reverse directions and applying non-equilibrium statistical mechanics one can estimate the free energy differences more efficiently. A critical component of alchemical transformations, whether equilibrium or nonequilibrium, is the definition and variation of the lambda function that controls the mixing of alchemical energy terms in the Hamiltonian. Linear variation of lambda is straightforward but may not be optimal, as it can lead to inadequate sampling of important configurations or the system getting trapped in nonreliable states. Nonlinear lambda schedules can be employed to mitigate these issues by adjusting the rate at which interactions are turned on or off during the transformation. Importantly, the lambda parameter can be separated into several named lambda functions that modulate specific interactions separately.

Based on the set of chemical species, a perturbation network may be designed in such a way that defines optimal transformations between compounds by a set of connections between first and second molecules.

Additionally, accurate numerical estimation of free energy differences by traditional approaches often is hindered by issues introduced by finite sampling. This is most apparent when applying the techniques relying on nonequilibrium dynamics and attempting to recover equilibrium properties such as ΔF by exponential reweighting. For example, one can derive the free energy difference by applying NEQS and recording the work distribution of the alchemical transformation as follows (assuming the probability distribution of work values had a density function P(W) with respect to Lebesgue measure):

$$exp ( - βΔ F ) = {\int }_{ℝ} {e}^{- β W} P ( W ) dW$$

From this follows that the most negative work values have the largest contribution to the estimate of the free energy. Unfortunately, these values typically correspond to the least observed low-energy states in the calculations, as shown in FIG. 1. This effect gives rise to large statistical uncertainty of the estimate and the problem commonly referred in the literature as the degeneracy of weights. One can alleviate this numerical instability by introducing many “fast-switching” trajectories to more efficiently sample the left tail of the work distribution. Alternatively, a “slow-switching” methodology with small switching rate might be used to maintain the system close to equilibrium along the transition path, effectively narrowing the work distribution and improving the final estimate. However, in the second approach, NEQS computational efficiency compares to that of equilibrium approaches. One can attempt to overcome the degeneracy of weights by applying a selection strategy to starting configurations and/or the transition paths, that would maximize the probability of performing a low-work switching simulation and minimize weights degeneracy.

The simulation setup may include conducting molecular dynamics simulations for each segment of the perturbation pathway. The simulation data is then analyzed to obtain relevant thermodynamic observations, such as potential energies, forces, and structural properties. Additionally, the obtained data may be used to compute the free energy difference between the first state and the second state. In some instances, the computed free energy may be further interpreted to validate experimental data or computation predictions, and/or to recognize limitations made in the perturbation network calculations.

While such a traditional approach may be used to generate a perturbation network, and consequently to compute free energy differences, such a traditional approach has certain limitations. For example, commonly used methods for designing a perturbation network are based on a generalized similarity metric. A similarity metric (e.g., Tanimoto and cosine metrics) can be computed using 1-dimensional (1D), 2-dimensional (2D), and/or 3-dimensional (3D) properties of molecular structures. For instance, a set of molecules can be clustered by maximum common substructure (MCS), in which the MCS represents the largest common structural motif shared by the molecules, by vector-based representations such as fingerprints, fragment descriptors, and embeddings produced with machine learning algorithms, or by shape similarity comparing 3D conformations of molecules. Such an approach, e.g. Lead Optimization Mapper (LOMAP) and High Information Mapper (HiMap), may not be effective in instances in which a perturbation network is needed for chemically distant compounds, particularly for compounds possessing different formal charges and compounds different in chemical structures but similar in charge volume distributions. For example, two known CAMKK2 inhibitors may include different central cores, in which the MCS-based methods may not consider such molecules as candidates for alchemical transformation in a substantial dataset, but these cores may be similar in terms of electronic properties and pharmacophore. Therefore, it would be advantageous to have a method of leveraging 3-dimensional (3D) features describing the electronic properties of molecules in creating perturbation networks

Additionally, the traditional approaches may face the problem of non-relevant microstates of simulated ligands. Being an endstate or intermediate during alchemical transformation, they might introduce an error to the binding free energy estimator by influencing the sampling-dependent accumulation of work increments. To mitigate this issue, it is imperative to eliminate any stage involving sampling high-energy states. Instead, the focus should be on deriving states exclusively from equilibrium ensembles of desired endstates. By ensuring that all microstates sampled during the simulation process are physically relevant and equilibrated, one can improve the accuracy of the binding free energy calculations and reduce the chances of introducing systematic errors.

Therefore, it would be advantageous to have a method of leveraging an approach of sampling protein and ligand conformations to reduce the problem of high-energy microstates.

### SUMMARY

In some embodiments, a computer-implemented method for generating an alchemical network (a graph) between states (graph nodes), e.g. the plurality of chemical species, is described. Each chemical species of the plurality considered, e.g. compounds A₁, A₂, . . . , A_{N}, may be described by a 3D representation or an ensemble of 3D representations. The alchemical transformations (graph edges) are considered for each pair of Aᵢ and Aⱼ from the plurality, and the pair-wise free energy differences for all compounds can be computed. Each edge of an alchemical network can be annotated by a value of the free energy difference. All values may be obtained by explicitly computing all pair-wise alchemical transformations, in total N(N−1)/2 transformations if the number of edges equals N. In this case, an alchemical network represents a complete graph with n nodes. All values may also be obtained by explicitly computing only a part of all pair-wise transformations (k<N(N−1)/2), and then restoring the rest of them, N(N−1)/2−k, using machine-learning algorithms applicable to graph problems. In this case, the alchemical network represents a connected graph with N nodes and k edges. The accuracy of the restored free energy differences depends on the topology of a connected graph. The method provides variations of alchemical network topology based on the molecular features of compounds A₁, A₂, . . . , A_{N}.

In some embodiments of the method, molecular features are employed to compute pair-wise distances, or similarity metrics, for N compounds. The features may include descriptors characterizing the 3D representations of compounds including but not limited to their electrostatic potential (ESP), volume and/or shape overlap ratio, root mean square deviation (RMSD) of matched atoms, and MCS. Then the similarity metrics between two compounds may be expressed as follows:

$$S = {\sum }_{i = 1}^{n} {w}_{i}^{′} {s}_{i} ,$$

$${s}_{k} = {S}_{ShapeESP} = \sqrt{{S}_{Shape} \times {S}_{ESP}} , k ∈ [ 1 , n ]$$

$${w}_{i}^{′} = \frac{{w}_{i}}{{\sum }_{j = 1}^{n} {w}_{j}}$$

where S represents a final weighted similarity metric score between ligand A (e.g., first ligand) and ligand B (e.g., second ligand), w′ᵢ∈[0, 1] is the normalized weight assigned to the i-th metric, sᵢ∈[0, 1] is the individual score obtained from the i-th metric, S_{ShapeESP} is the combined score obtained from ESP similarity and shape similarity metrics.

In some embodiments of the method, the computed pair-wise distances based on ESP and other 3D features are used to cluster compounds and produce the variations of alchemical networks which are connected graphs. The method allows the production of alchemical networks for a plurality of compounds that do not share common sets of atoms but share similarities in electronic and charge distribution properties.

In some embodiments, a computer-implemented method for selecting conformational states of compounds and protein-compound complexes to carry out alchemical transformations is described. Compounds A₁, A₂, . . . , A_{N} may be considered to have the ability to bind to a target and may represent the nodes of an alchemical network. Then the alchemical transformation for each pair of Aᵢ and Aⱼ from the plurality considered may be simulated. The 3D conformational states of Aᵢ and Aⱼ, including their protein-compound complexes, are utilized to simulate the alchemical transformation between them. The accuracy and robustness of calculating the free energy difference for the transformation depend on the 3D conformational states of Aᵢ and Aⱼ employed for alchemical transformation. The method provides the strategy for sampling relevant conformational states of Aᵢ and Aⱼ from pre-generated conformational ensembles.

In the above embodiments, conformational ensembles for compounds A₁, A₂, . . . , A_{N} can be generated using molecular and/or quantum mechanics simulations (MM/QM), Monte-Carlo simulations, or simulations computed with artificial neural networks. The multiple pairs of states of Aᵢ and Aⱼ can be selected to carry out alchemical transformations and compute the value of free energy difference. The method allows the sampling of conformational states in a way that takes into account a state of a molecular system at the beginning of alchemical simulations, for instance, the side chain conformers of amino acid residues surrounding compound Aⱼ, and thus preserving reliable microstates and ensuring an optimal transition pathway along with reproducible free energy estimates. The criteria for selecting a pair of states for compounds Aᵢ and Aⱼ can be geometry- and/or energy-based.

In some embodiments, one or more non-transitory computer readable media are provided that store instructions that in response to being executed by one or more processors, cause a computer system to perform operations, the operations comprising performing the computer methods described herein for generating a diagram of changes.

In some embodiments, a computer system can include: one or more processors; and one or more non-transitory computer readable media storing instructions that in response to being executed by the one or more processors, cause the computer system to perform operations, the operations comprising performing the methods described herein for generating a diagram of changes or perturbation graph.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 shows the perturbation equation integrand as a product of P(W) and exp(−betaW) with work degeneracy region filled grey.

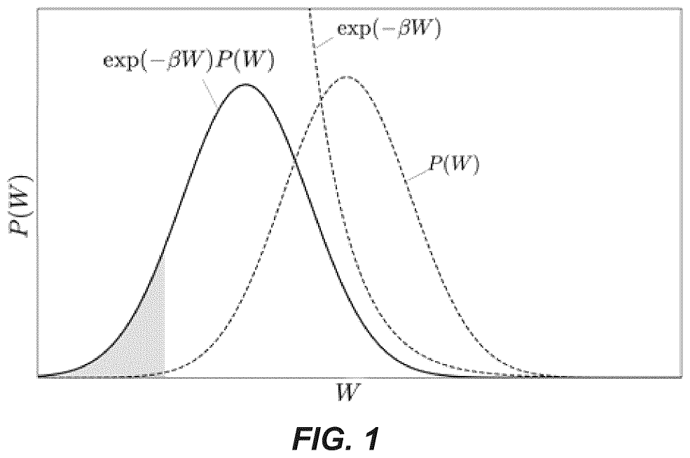

FIGS. 2A-B show examples of compounds having different chemical structures.

FIG. 3A shows an example superposition of two different molecules.

FIG. 3B shows an example of an alchemical network.

FIG. 4 is a flow chart of an example method of building a perturbation graph.

FIG. 5 shows result of sampling a ghost ligand microstate from the gas phase-space.

FIG. 6A is a flow chart of an example method of performing an alchemical transformation using the microstate sampling method.

FIG. 6B is a flow chart of an example method of analyzing an alchemical transformation.

FIG. 7 shows an example computing device.

FIG. 8 illustrates a schematic example of a computing platform in accordance with a diagram generating protocol.

FIG. 9 illustrates a schematic example of a computing platform in accordance with an alchemical transformation analysis.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The methodologies provided herein can be performed on a computer or in any computing system. In some embodiments, the computer can include generative adversarial networks that are adapted for conditional generation of objects (e.g., generated objects), when a known external variable, such as the condition/property, influences and improves generation and decoding. When data consists of pairs of complex objects, e.g., a supervised dataset with a complex condition/property for a molecule, the computing system can create a generated complex object (e.g., molecules) that is similar to the provided complex object (e.g., provided molecule) of the data that satisfies the complex condition/property (e.g., biological activity, physiochemical property, etc.) of the data. The computing system can process the models described herein that are based on the adversarial autoencoder architecture that can learn three latent representations: (1) object/molecule only information; (2) condition/property only information, and (3) common information between the object/molecule and the condition/property. The model can be validated or trained with a dataset of molecules with a high objective function for the property, where common information is a digit, and then apply the training to a practical problem of generating fingerprints of molecules with desired properties. In addition, the model is capable of metric learning between objects and conditions without negative sampling.

The condition usually represents a target variable, such as a class label in a classification problem, which represents one or more desired properties. In an example, the condition “y” is a complex object itself, such as biological activity. For example, drug discovery is used to identify or generate specific molecules with a desired action on human cells (e.g., such a property), or molecules that bind to some protein. In both cases, the condition (e.g., protein binding) is at least as complex as the object (e.g., a candidate molecule for a drug) itself. The protocols described herein can be applied to any dataset of object/property pairs (x, y). When a computing process operates with the models described herein, the computer can extract common information from the object and the condition/property and rank generated objects by their relevance to a given condition and/or rank generated conditions by their relevance to a given object.

The model includes the encoders performing a decomposition of the object data and condition data to obtain the latent representation data. The latent representation data is suitable for conditional generation of generated objects and generated conditions/properties by the generators and may also be suitable for use in metric learning between objects and conditions.

As used herein, the model includes encoders Ex and Ey, a generators Gx and Gy (i.e., decoders), and “x” is the object molecule, “y” is the condition/property, and all z correspond to the latent representations produced by the encoders. The model can be applied to a problem of mutual conditional generation of “x” and “y” given a dataset of pairs (x, y). Both x and y can be assumed to be complex, each containing information irrelevant for conditional generation of the other.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Generally, the present technology relates to computing free energy differences associated with chemical transfer processes. Particularly, the present technology may provide a method for designing a perturbation network and a modification of an alchemical transformation protocol.

In some embodiments, the perturbation network may be designed based on 3D features of molecules or substances. For example, the 3D features may include 3D shapes and spatial electrostatic potential (ESP), among others. The ESP may represent distribution of electric charges within a molecule or a system, which may provide insights into the distributions of electron density. While 2D representation and/or features of molecules provide information about molecular connectivity and certain properties, 2D representations are limited with respect to spatial information, conformational flexibility, intermolecular interactions, stereochemistry, etc. Such limitations may cause perturbation networks designed based on 2D representations to be limited.

In some embodiments, the 3D features of the molecules may be used to design the perturbation networks. For example, a list of substances may be obtained. In some embodiments, the substances may include molecules, small-molecule ligands, etc. Pair-wise distances between the substances may be calculated based on the 3D shapes and ESP of the substances.

In some embodiments, alchemical transformation process may be modified such that microstates of a chemical compound may be sampled from phase spaces relevant to the chemical compound. For example, an alchemical transformation may involve a transformation from a first compound to a second compound. Conformations of the second compound may be sampled in a phase space similar to the phase space relevant to the first compound, such that environment at the beginning of the simulation may be taken into consideration during the sampling process.

### Alchemical Network

Traditional approaches to designing perturbation graphs (alchemical networks) are limited with respect to chemically different compounds. For example, chemically different compounds may have different molecular structures, sizes, and functional groups. Building an alchemical network based on the traditional approaches (e.g., based on the MCS) may be limited due to various factors. For example, the MCS approach relies on identifying common substructures among different compounds to infer potential similarities. Chemically different compounds may share limited structural similarity, in which the identification of common substructures may be challenging. Additionally, the MCS approach does not directly observe or identify functional similarities or differences. Chemically different compounds may exhibit similar pharmacological effects by targeting common biological pathways or protein families. The MCS approach may overlook such functional relationships in designing the perturbation networks.

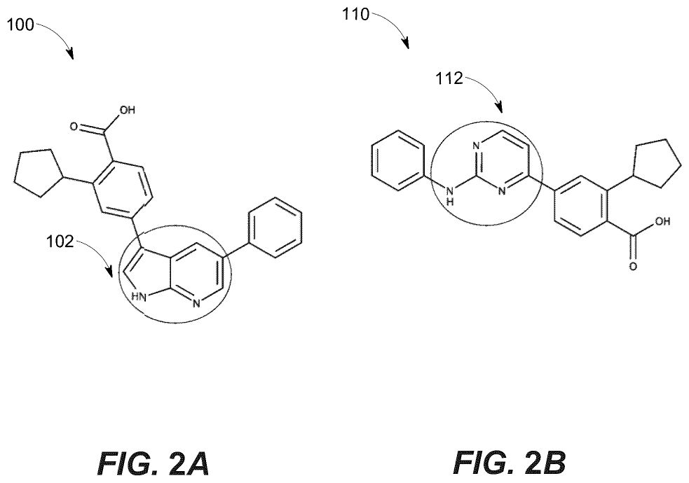

For example, FIGS. 2A and 2B illustrate two known calcium/calmodulin-dependent protein kinase kinase 2 (CAMKK2) inhibitors. CAMKK2 inhibitors may include compounds that target and inhibit the enzymatic activity of CAMKK2. However, the inhibitors may have varying chemical structures. For example, a first inhibitor 100 of FIG. 2A and a second inhibitor 110 of FIG. 2B may contain different central cores. For example, the first inhibitor 100 may include a first central core 102 and the second inhibitor 110 may include a second central core 112.

Traditional methods of designing alchemical networks based on the MCS approach, a structural similarity, and/or a shape similarity may overlook the similarities of electronic properties and/or charge distributions between the first inhibitor 100 and the second inhibitor 110 based on structural differences. Such overlook may cause the dismissal of such molecule pairs as candidates for alchemical transformation.

Contrastingly, according to at least one embodiment of the present disclosure, 3D electronic and charge features of substances and/or molecules may be considered in designing the perturbation networks. For example, the shapes and spatial electrostatic potential (ESP) of the molecules may be considered. The shapes of the molecules may include 3D shapes of the molecules. For example, the shapes may represent spatial arrangements of atoms in three dimensions, including bond angles and bond lengths. The 3D shapes of the molecules may affect the molecule's physical and chemical properties.

The ESP may represent the distribution of electric charge across the molecular surface. The distribution may be determined based on the arrangement of atoms and respective electron densities within the molecules. In some embodiments, the ESP of the molecules may be represented visually. For example, the regions of the molecular surfaces may be represented using different indicators (e.g. colors) based on the electrostatic potential.

In some embodiments, the ESP may be used to determine similarities between chemically different compounds. For example, the map of Coulomb potentials for each compound may be calculated. The ESP may be represented as a grid of points surrounding each molecule, in which each point represents a corresponding electric potential. The grid-based representations of different compounds may be used to compare the ESP between different molecules. The overlap integral as well as self-overlap integrals of ESP may further represent similarities between the compounds. In some embodiments, the similarities between the ESP grids may be determined using any suitable methods and/or similarity metrics. For example, the similarities may be measured using correlation coefficients, Euclidean distance, Cosine similarity, Carbo similarity, Tanimoto metric, among others.

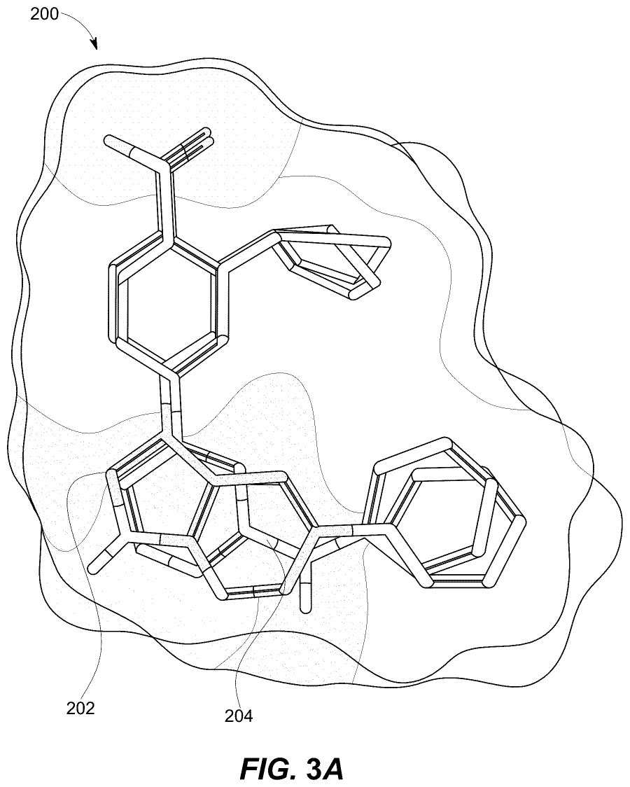

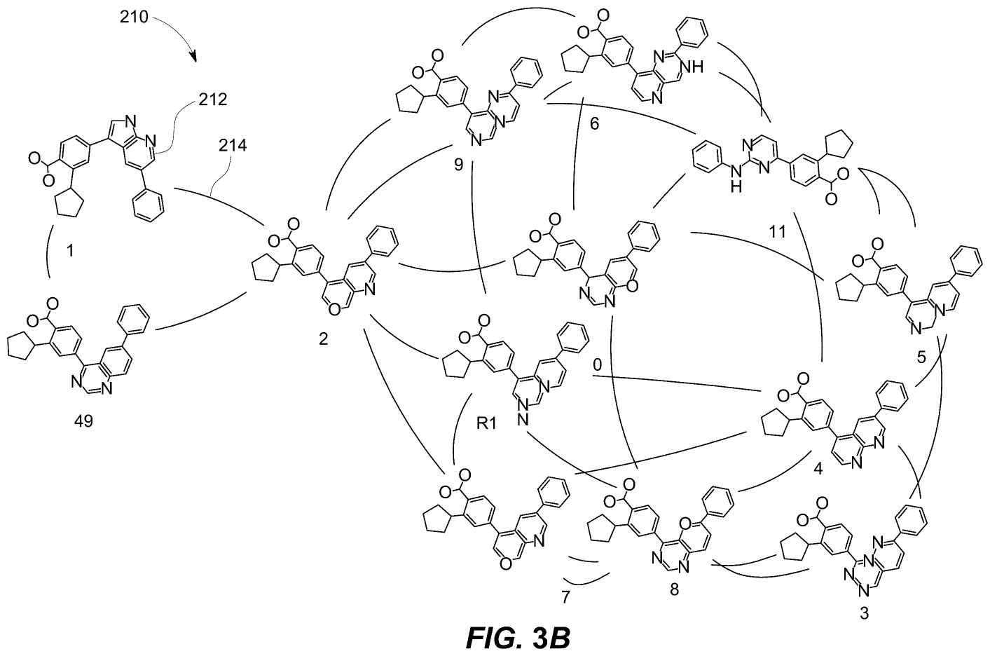

FIG. 3A illustrates a superposition 200 of the two different molecules, in accordance with one or more embodiments of the present disclosure. For example, the superposition 200 may represent alignment between a first inhibitor 202 and a second inhibitor 204. In some embodiments, the first inhibitor 202 may correspond to the first inhibitor 100 of FIG. 2A and the second inhibitor 204 may correspond to the second inhibitor 110 of FIG. 2B. The superposition 200 may align the structures of the first inhibitor 202 and the second inhibitor 204 such that similarities and differences between the first inhibitor 202 and the second inhibitor 204 may be observed.

In some embodiments, the superposition 200 may be determined based on the shapes and ESP of the first inhibitor 202 and the second inhibitor 204. For example, the structures of the first inhibitor 202 and the second inhibitor 204 may be analyzed to determine similar structures and/or regions with similar ESP. Unlike the 2D comparison of the first inhibitor 202 and the second inhibitor 204, the comparison based on the shape and the ESP indicates a degree of similarity between the first inhibitor 202 and the second inhibitor 204. Based on such similarity a pair of molecules 202 and 204 may be considered for alchemical transformation.

In some embodiments, alchemical networks designed based on the molecular shapes and ESP may be used with respect to scaffold hopping. For example, the alchemical networks may be used to identify chemical compounds that have different core scaffolds or molecular frameworks compared to a reference compound while retaining or improving desired biological activities. For example, comparison of different molecules based on shape and ESP may allow identification of alchemical transformations that may include different core scaffolds while retaining general biological properties. Such an approach may improve and/or overcome certain limitations of the MCS-based approaches for building alchemical networks.

FIG. 3B illustrates an example alchemical network 210. The alchemical network 210 may include nodes 212 and edges 214, in which the nodes 212 represent compounds, and the edges 214 represent transitions from one compound to another compound. For example, in instance in which the compounds are molecules undergoing a chemical reaction, the graph may show the transitions from reactants to products, represented as the nodes 212 and the edges 214.

In some embodiments, the nodes 212 may be represented using unique identifiers (e.g., compound IDs or names) or using structural properties (e.g., molecular fingerprints, chemical structures, etc.). Additionally or alternatively, the nodes 212 may include information regarding chemical properties of the compounds.

In some embodiments, the edges 214 may represent relationships or alchemical transformations between compounds. For example, an edge between a first compound and a second compound may indicate that there is an alchemical transformation or interaction between the first compound and the second compound. In some embodiments, the edges 214 may be weighted. For example, the edges 214 may be associated with weights, in which the weights represent strength and/or magnitude of the alchemical transformation or interaction between the compounds. In some embodiments, the weights may be represented quantitatively, such as similarity scores. In other embodiments, the edges 214 may be unweighted, in which the edges 214 do not have weights, or all edges 214 have the same weight.

Additionally or alternatively, in some embodiments, the edges 214 may have directionality. For example, the edges 214 may be configured to represent direction of the alchemical transformation between compounds. For example, a directed edge from the first compound to the second compound may indicate that the first compound perturbs into the second compound.

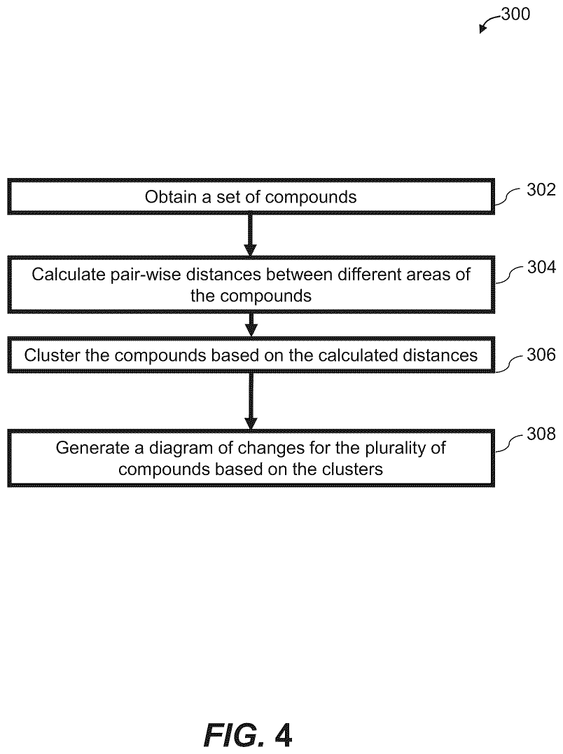

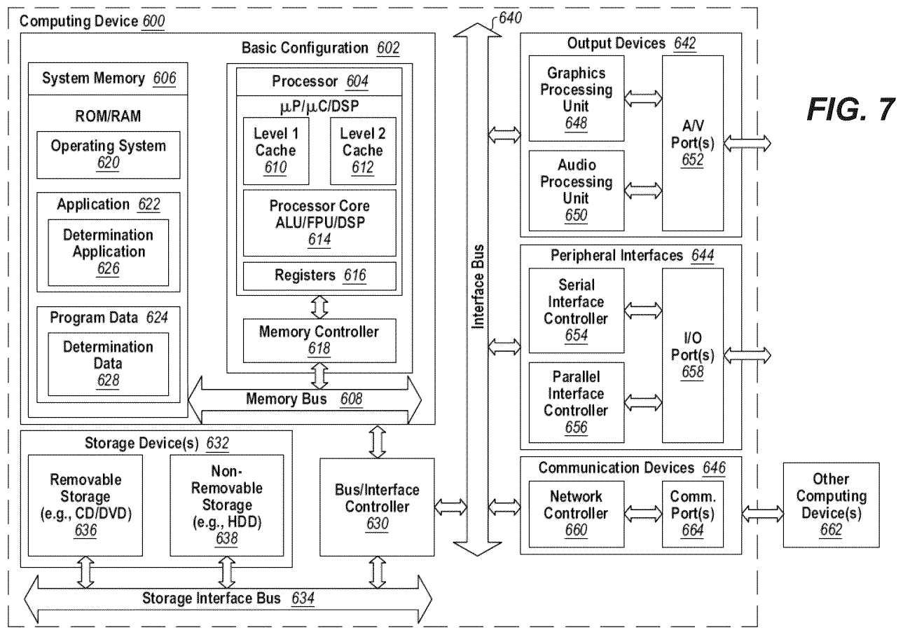

FIG. 4 includes a flowchart illustrating an example method 300 of generating a diagram or an alchemical network, in accordance with one or more embodiments of the present disclosure. One or more operations of the method 300 may be implemented using a suitable computer or processor such as the computing device 600 of FIG. 7. Although illustrated as discrete steps, various steps of the method 300 may be divided into additional steps, combined into fewer steps, or eliminated, depending on the desired implementation. Additionally, the order of performance of the different steps may vary depending on the desired implementation.

The method may include begin at block 302. At block 302, a set of molecules and/or compounds may be obtained. In some embodiments, N number of compounds may be obtained. In some embodiments, the compounds may include small-molecule ligands. In some embodiments, data associated with the set of compounds may be obtained and/or gathered. For example, the data may include chemical structures, biological activities, and other computational data associated with each compound of the set of compounds.

In some embodiments, the obtained data may be processed such that the data may be used to design the alchemical network. For example, the data corresponding to individual compounds of the set of compounds may be analyzed such that duplicates and errors may be removed and/or corrected. Additionally or alternatively, irrelevant or low-quality data points or compounds may be removed. In some embodiments, the removed compounds may be replaced with other compounds including higher-quality data.

In some embodiments, each compound may have a first state and a second state. In some embodiments, the first state may represent a reference thermodynamic state and the second state may correspond to a target thermodynamic state of the compound. For instance, the first state and the second state may represent two different binding modes of the compound with a protein target.

At block 304, pairwise or comparative distances between different areas of the compounds may be calculated based on the physical configurations and electrostatic potentials of the compounds. For example, similarity metrics based on the shapes and electrostatic potentials may be computed between different regions of the compounds. In instances in which the compounds are molecules, the different regions may refer to different atoms or groups of atoms within the molecule. In some embodiments, the similarity metrics may include distance metrics that may be used to calculate pairwise distances between the pairs of compounds represented as distances. For example, shape-based metrics (e.g., comparing overall shapes of molecules, regardless of atomic compositions or arrangements) and electrostatic potential matching (e.g., comparing spatial distribution of electrostatic potentials on the molecular surfaces) may be used. In some embodiments, any other suitable distance metrics may be used, such as root mean square deviation (RMSD), Tanimoto distance, contact-based metrics, or functional group matching.

In some embodiments, the distances may be calculated for compound in both the first state and the second state. For example, the distances between different regions of within the compound may be calculated with respect to the compound in the first state and in the second state. In some embodiments, the distances may be determined using a machine learning algorithm.

At block 306, the compounds may be clustered based on the calculated distances. For example, the compounds may be grouped together based on similarities and/or dissimilarities with respect to perturbation profiles of the compounds. In these and other embodiments, the perturbation profiles may represent how each compound perturbs a biological system and/or target of interest. The profile may provide information on the effects and/or interactions of the compounds within the system. For example, the perturbation profiles may include information regarding the calculated distances between different areas of each compound. In these and other embodiments, the compounds may be clustered such that compounds with similar calculated distances may be clustered together.

In some embodiments, a distance matrix may be generated based on the calculated distances. The distance matrix may include elements that represent the distances between different areas within the compounds. The distance matrix may be used to compare the distances such that degrees of similarities and differences may be determined.

In some embodiments, the compounds may be clustered based on the calculated distances using a clustering algorithm suitable for clustering compounds based on the distances. For example, k-means clustering, density-based clustering, hierarchical clustering, among others, may be used to cluster the compounds. In these and other embodiments, certain parameters may be specified based on the types of clustering algorithms used. For example, a number of clusters or hierarchical structures may be specified.

At block 308, a diagram of changes for the plurality of compounds may be generated based on the clusters. In some embodiments, the diagram may include an alchemical graph. The alchemical graph may include nodes and edges, in which the nodes represent compounds, and the edges represent transitions from one compound to another compound. For example, in instance in which the compounds are molecules undergoing a chemical reaction, the graph may show the transitions from reactants to products, represented as nodes and edges.

In some embodiments, the nodes may be represented using unique identifiers (e.g., compound IDs or names) or using structural properties (e.g., molecular fingerprints, chemical structures, etc.). Additionally or alternatively, the nodes may include information regarding chemical properties of the compounds.

In some embodiments, the edges may represent relationships or alchemical transformations between compounds. For example, an edge between a first compound and a second compound may indicate that there is an alchemical transformations or interaction between the first compound and the second compound. In some embodiments, the edges may be weighted. For example, the edges may be associated with weights, in which the weights represent strength and/or score of the alchemical transformation or interaction between the compounds. In some embodiments, the weights may be represented quantitatively, such as similarity scores. In other embodiments, the edges may be unweighted, in which the edges do not have weights, or all edges have the same weight.

Additionally or alternatively, in some embodiments, the edges may have directionality. For example, the edges may be configured to represent direction of the alchemical transformation between compounds. For example, a directed edge from the first compound to the second compound may indicate that the first compound perturbs the second compound.

In some embodiments, a number of edges in the alchemical networks may be limited to equal to or less than N(N−1)/2, in which N represents the number of compounds represented using the graph. Preferably, the number of the edges may be equal to N*ln(N). In these and other embodiments, specifically limiting the number of edges may help preserving stable precision. For example, limiting the number of edges may limit the complexity of the graph which may help focusing on the most relevant alchemical transformations or interactions between the compounds.

In instances in which the alchemical graph includes more edges than the limit (e.g., N(N−1)/2 or N*ln(N)), certain edges may be removed from the alchemical graph. For example, in instances in which the edges are weighted, the edges with the least weight may be removed until the number of edges is within the limit.

### Conformation Sampling Protocol

An alchemical transformation may refer to a technique used to model gradual transition of a first chemical compound to a second chemical compound. Such a process may be used to analyze processes such as ligand binding, protein-ligand interactions, and chemical reactions. For example, an alchemical transformation may include a transformation from a first ligand to a second ligand. A transformation pathway, specifying how the system transitions from the first ligand to the second ligand, may be established. Additionally, a continuous parameter that controls degree of transformations between the first ligand and the second ligand may be defined.

Based on the transformation pathway, the continuous parameter, the first ligand, and the second ligand, different conformations along the transformation pathway may be sampled. For example, different conformations of the second ligand may be sampled.

Commonly, the sampling of the ghost ligand is performed in the decoupled phase. Decoupled phase may represent a condition where molecules are simulated in the absence of a condensed phase and often without any explicit solvent molecules or/and external fields. In the decoupled phase, the molecules may be allowed to move and interact freely in a simulated vacuum environment. Sampling ligands in the decoupled phase may include computational procedure in which the dynamic behavior of ligand molecules is investigated in isolation, without considering interactions with other molecules or a solvent environment. Such sampling may be useful for studying isolated molecules and their conformational flexibility as represented by visited microstates.

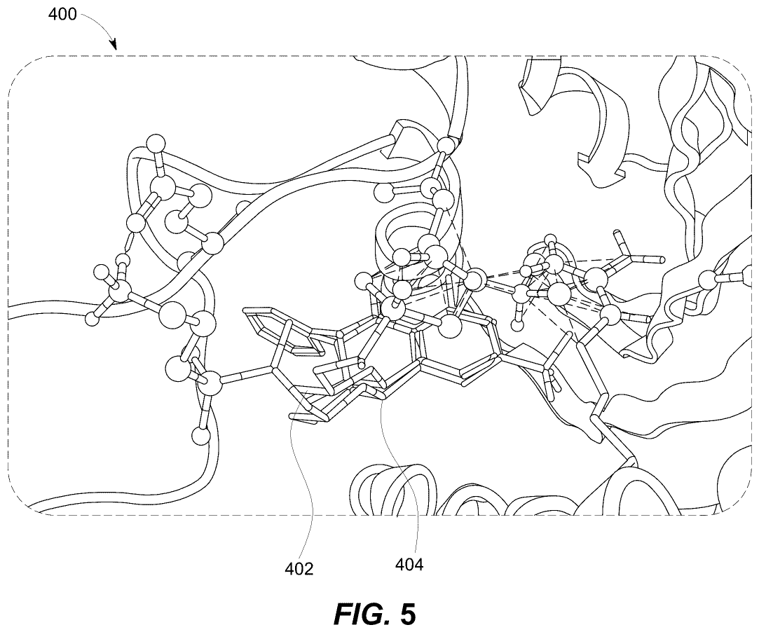

For example, FIG. 5 illustrates a result of sampling a ghost ligand conformer from the gas phase-space. For example, a second ligand 402 may be a target thermodynamic state of an alchemical transformation from a first ligand. The second ligand 402 may include a rigid core common to the first ligand as illustrated. However, the second ligand 402 may also include a flexible chain that may have multiple conformations and sampling the second ligand 402 in the gas phase may result in a pose having severe clashes with a protein target. For example, FIG. 5 illustrates the second ligand 402 having sever clashes with a protein 404. Such clashes may lead to significant errors in energy estimation and free energy calculation.

According to one or more embodiments of the present disclosure, microstates of a second ligand, such as the second ligand 402 of FIG. 5, may be sampled from relevant phase space. The relevant phase space may refer to a subset of total phase space that is pertinent to the phenomena of interest. With respect to ligands, the relevant phase space may include a range of conformations that the ligand may adopt through interacting with binding site or solvent molecules.

In some embodiments, sampling the microstates of the second ligand may be performed from an independent equilibrium simulation. An independent equilibrium simulation may refer to conducting multiple simulations under different initial conditions or settings, each simulation exploring equilibrium behavior of a complex.

As an example, independent equilibrium simulations of a complex may involve, preparing an initial configuration of the complex (e.g., a protein-ligand complex). For example, the ligand may be placed in the binding site of the protein and the system may be solvated in an appropriate solvent model. Each simulation may start from at least slightly different initial configuration. In some instances, the different initial configurations may be formed through random perturbations to the initial positions of the atoms. Then, multiple simulations may be run starting from different initial configurations. For example, the system may be allowed to evolve until equilibrium (e.g., properties of interest no longer exhibit significant fluctuations) is reached.

In some embodiments, to preserve reliable sampling of ghost ligand conformations, a special nonlinear schedule for the alchemical mixing of nonbonded interactions can be applied. Conventional approaches incorporate so-called lambda components, which are parameters responsible for mixing various intra- and intermolecular interactions into the Hamiltonian of the simulated system of interest. These lambda components are defined as sets of values within the [0, 1] interval and dictate the progression of the alchemical transformation.

In preferred embodiments, nonbonded interactions—including van der Waals and/or electrostatic interactions—are engaged using a nonlinear lambda variation that is a function of transformation time and is proportional to an arcsine function or a smoothstep function. By defining lambda as a time-dependent function, the method allows for more controlled and efficient sampling during the alchemical transformation. Utilizing these functions for lambda scheduling allows for more controlled and efficient sampling during the transformation. Mathematically, in preferred embodiments lambda λ(t) can be defined as but not limited to:

$${λ}_{nonbonded} \approx {( \frac{t}{T} )}^{2} ( 3 - 2 \frac{t}{T} ) \qquad Equation 1$$

$${λ}_{nonbonded} \approx {sin}^{- 1} ( \frac{t}{T} ) \qquad Equation 2$$

where t is the current simulation time and T is the total simulation time. The use of an arcsine function implies that the lambda values change rapidly at both the beginning and end of the transformation, with slower variation in the middle stages. This time-dependent approach ensures that the system moves quickly away from high-energy states associated with ghost ligands at the start and smoothly approaches the reliable state at the end, thus improving the reliability of the simulation.

Employing a smoothstep function of time corresponds to a slow variation of lambda values at the beginning and end of the transformation, with faster changes in the intermediate stages. In certain embodiments it ensures that lambda changes gradually at the start and end of the simulation, allowing the system to adequately sample the reference and target states over time. By modulating the rate of change in lambda values as a function of time, the smoothstep function helps maintain equilibrium conditions throughout the transformation.

By carefully selecting these nonlinear, time-dependent lambda schedules, the method ensures reliable sampling of microstates throughout the alchemical process. This temporal modulation not only preserves the physical relevance of the sampled states but also contributes to an optimal transition path, ultimately leading to more accurate and reproducible free energy estimates. Moreover, these nonlinear scheduling techniques mitigate issues associated with abrupt changes in interactions that are common in linear lambda schedules. By smoothing out the transitions over time and concentrating sampling efforts where they are most needed, the method enhances the efficiency and reliability of alchemical free energy calculations. It effectively reduces the potential for systematic errors by ensuring that all significant states are adequately sampled throughout the simulation period, particularly in regions where the system is sensitive to changes in interactions.

In some instances, relative probabilities of finding systems in different states with respect to each ligand's energy levels may be represented as a partition function ratio. With respect to ligands binding to a protein, the partition function ratio may provide insights into the thermodynamic equilibrium between different binding configurations.

In some embodiments, the partition function ratio may be represented as Equation 3.

$${e}^{- βΔ {G}_{site , AB}} = \frac{{\int }_{Γ} {e}^{- β U ( q ; {λ}_{B} )} {I}_{B} ( q ) dq}{{\int }_{Γ} {e}^{- β U ( q ; {λ}_{A} )} {I}_{A} ( q ) dq} \qquad Equation 3$$

Where ΔG_{site,AB} represents difference in free energy between ligand A (e.g., first ligand) and ligand B (e.g., second ligand), β=k_{B}T1 (product of Boltzmann's constant and temperature), U(q;λ_{B}) and U(q;λ_{A}) representing potential energy functions for ligand B and ligand A, respectively, I_{B}(q) and I_{A}(q) representing indicator functions and the integrals representing integrals over the configuration space of ligand B and ligand A. I_{B}(q) and I_{A}(q) may equal to 1 for bound and 0 for unbound ligand states.

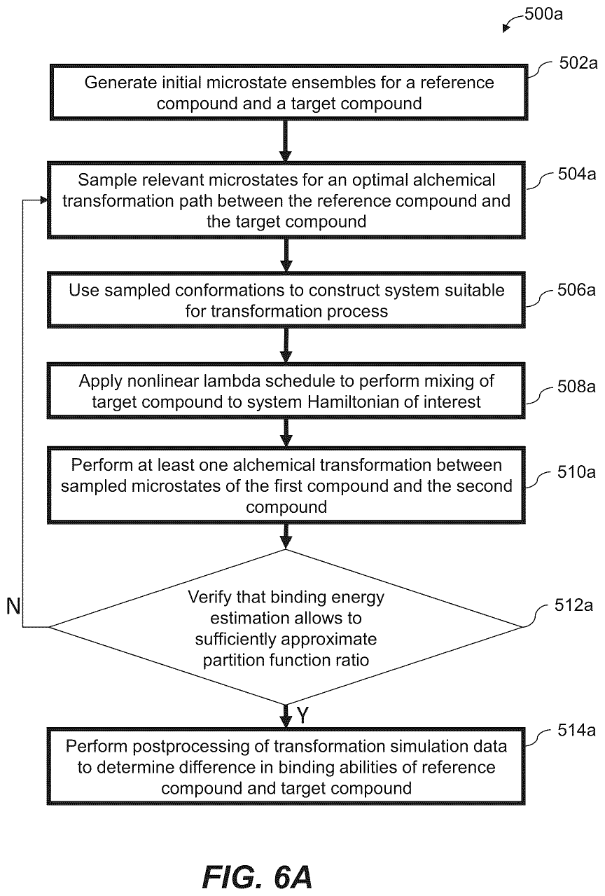

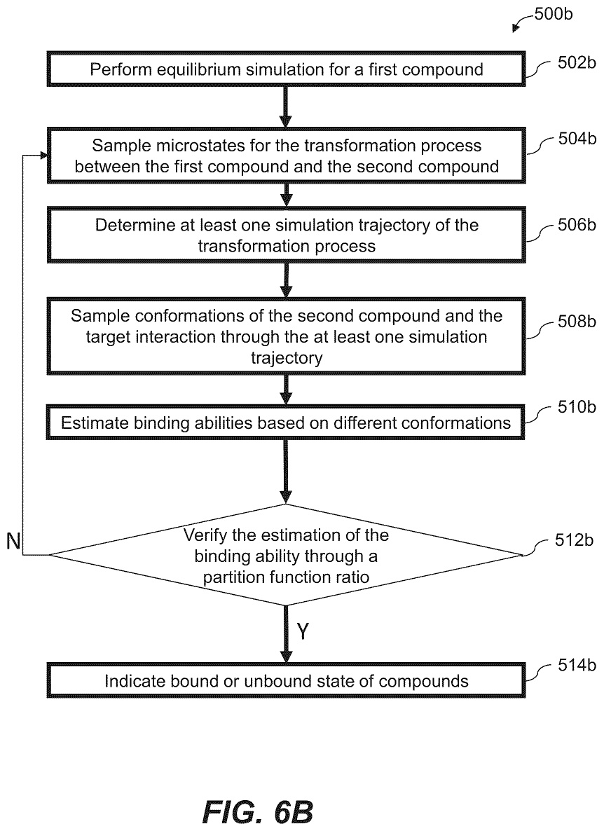

FIG. 6A illustrates an example method 500a of performing an alchemical transformation, arranged in accordance with at least one embodiment of the present disclosure. One or more operations of the method 500a may be implemented using a suitable computer or processor such as the computing device 600 of FIG. 7. Although illustrated as discrete steps, various steps of the method 500a may be divided into additional steps, combined into fewer steps, or eliminated, depending on the desired implementation. Additionally, the order of performance of the different steps may vary depending on the desired implementation.

In some embodiments, the method 500a may include a block 502a. At block 502a, computational simulation for a reference compound and a target compound of a set of compounds may be carried out. In some embodiments, the computational simulation may include using molecular dynamics simulations to predict how the reference compound and the target compound interact with a specific target or protein. For example, given the reference compound and the specific target, the computational simulation may be used to predict how the first compound may interact with the specific target. In some embodiments, the simulation may generate a set of potential binding poses. In some embodiments, the first compound may reflect a reference thermodynamic state or a starting point of an alchemical transformation. In some embodiments, the second compound may reflect a target thermodynamic state or an ending state of an alchemical transformation. In some embodiments, the simulation may generate a trajectory that represents the movement of the compound and the specific protein over time, which may be used to estimate the binding ability of the compound. In some embodiments, the set of compounds including the reference compound and the target compound may include drugs.

In some embodiments, the simulation may be done using any suitable simulation methods such as molecular dynamics simulation, Monte Carlo simulation, quantum mechanics simulation, simulations employing artificial neural networks, among others.

At block 504a, microstates for the alchemical transformation process between the reference compound and the target compound may be sampled. One or more microstates of each compound within a corresponding phase may be sampled through the simulation trajectory or pathway. For example, each compound may have different conformations in interacting with the specific protein or solvent. In some embodiments, the microstates may represent intermediate states during the transformation process from the reference compound to the target compound. For example, a computational simulation may be run using the reference compound and the target compound to predict the difference in the binding free energy. In some embodiments, multiple trajectories of the transformation simulation may be determined between the reference compound and the target compound. For example, the reference compound could transform into the target compound via different pathways.

At block 506a, the sampled microstates of the reference compound and the target compound may be utilized to construct a system suitable for the transformation. For example, a hybrid system may be generated using the sampled microstates of the reference compound to the target compound.

At block 508a, a lambda schedule may be applied to the sampled microstates of the target compound to perform mixing with the system Hamiltonian. The lambda schedule may be represented by a nonlinear mathematical function.

At block 510a, at least one alchemical transformation between the reference compound and the target compound may be performed. For example, at least one simulation including one or more microstates of the reference compound and the target compound may be determined and/or selected.

At block 512a, the validation that binding energy estimation allows a sufficient approximation of a partition function may be done. For example, the approximation of the partition function ratio such as Equation 3 may be verified.

In response to the estimation failing to be verified, the microstates for the transformation process between the first compound and the second compound may be redetermined, returning to block 504a.

In response to the estimation being verified, at block 514a, the difference in binding free energy between the reference compound and the target compound may be computed and reported.

Modifications, additions, or omissions may be made to the computing system 600 without departing from the scope of the present disclosure. For example, in some embodiments, the computing system 600 may include any number of other components that may not be explicitly illustrated or described.

FIG. 6B illustrates an example method 500b of analyzing an alchemical transformation, arranged in accordance with at least one embodiment of the present disclosure. One or more operations of the method 500b may be implemented using a suitable computer or processor such as the computing device 600 of FIG. 7. Although illustrated as discrete steps, various steps of the method 500b may be divided into additional steps, combined into fewer steps, or eliminated, depending on the desired implementation. Additionally, the order of performance of the different steps may vary depending on the desired implementation.

In some embodiments, the method 500b may include a block 502b. At block 502b, equilibrium simulation for a first compound of a set of compounds may be performed. For instance, a computational model for the first compound may be performed. In some embodiments, the computational model may include using computer-based simulation to predict how the first compound interacts with a specific target or protein. For example, given the first compound and the specific target, the computational model may be used to predict how the first compound may interact with the specific target. In some embodiments, the simulation may generate a set of potential binding sites and the strength of interaction between the first compound and the target. In some embodiments, the first compound may reflect a reference thermodynamic state or a starting point of an alchemical transformation. In some embodiments, the simulation may generate a trajectory that represents the movement of the first compound and the specific protein over time, which may be sued to estimate the binding ability of the first compound. In some embodiments, the set of compounds including the first compound may include drugs.

In some embodiments, the simulation may be done using any suitable simulation methods such as molecular dynamics simulation, Monte Carlo simulation, quantum mechanics simulation, among others.

At block 504b, microstates for the alchemical transformation process between the first compound and the second compound may be sampled. In some embodiments, the microstates may represent intermediate states during the transformation process from the first compound to the second compound. For example, a computational simulation may be run using the first compound and the second compound to predict the transformation process. In some embodiments, multiple simulation trajectories may be determined between the first compound and the second compound. For example, the first compound could transform to the second compound via different pathways.

At block 506b, at least one simulation trajectory or pathway may be determined from the transformation process. For example, at least one trajectory including one or more microstates may be determined and/or selected.

At block 508b, one or more conformations of the second compound and the target interaction may be sampled through the simulation trajectory or pathway. For example, the second compound may have different conformations in interacting the specific target or protein.

In some embodiments, the conformations may be determined based on simulation of the second compound. In some embodiments, the simulation may be performed in a similar as the simulation of the first compound. In some embodiments, the simulation of the second compound may be done from relevant phase space. In these and other embodiments, the relevant phase space may be determined based on conformational space, energy landscape, and/or quantum state space of the first compound and/or the second compound. In some embodiments, the simulation of the second compound may be done from equilibrium similar to the first compound.

At block 510b, binding abilities of different conformations of the second compound may be estimated. In some embodiments, the binding abilities may be more precisely estimated for simulation of the second compound from relevant phase space or equilibrium compared to in gas phase. Sampling the conformations of the second compound from gas-phase space may result in utilizing not one or more microstates not relevant for estimating the binding ability or for estimating a partition function ratio, which may lead to significant errors in estimations.

At block 512b, the estimation of the binding ability may be verified through a partition function ratio. For example, using the partition function ratio, the estimation may be verified. For example, the partition function ratio may provide a ration that represents the binding ability of the first compound and the second compound which may be used to verify the estimation.

In response to the estimation failing to be verified, the microstates for the transformation process between the first compound and the second compound may be redetermined, returning to block 504b.

In response to the estimation being verified, at block 514b, bound or unbound states of compounds may be indicated. For example, a binary signal for ligand binding may be used to indicate the bound or unbound states of the compounds. For example, 1 may represent a bound state and 0 may represent unbound state.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only, and is not intended to be limiting.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

FIG. 7 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method that can include: providing a dataset having object data for an object and condition data for a condition; processing the object data of the dataset to obtain latent object data and latent object-condition data with an object encoder; processing the condition data of the dataset to obtain latent condition data and latent condition-object data with a condition encoder; processing the latent object data and the latent object-condition data to obtain generated object data with an object decoder; processing the latent condition data and latent condition-object data to obtain generated condition data with a condition decoder; comparing the latent object-condition data to the latent-condition data to determine a difference; processing the latent object data and latent condition data and one of the latent object-condition data or latent condition-object data with a discriminator to obtain a discriminator value; selecting a selected object from the generated object data based on the generated object data, generated condition data, and the difference between the latent object-condition data and latent condition-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

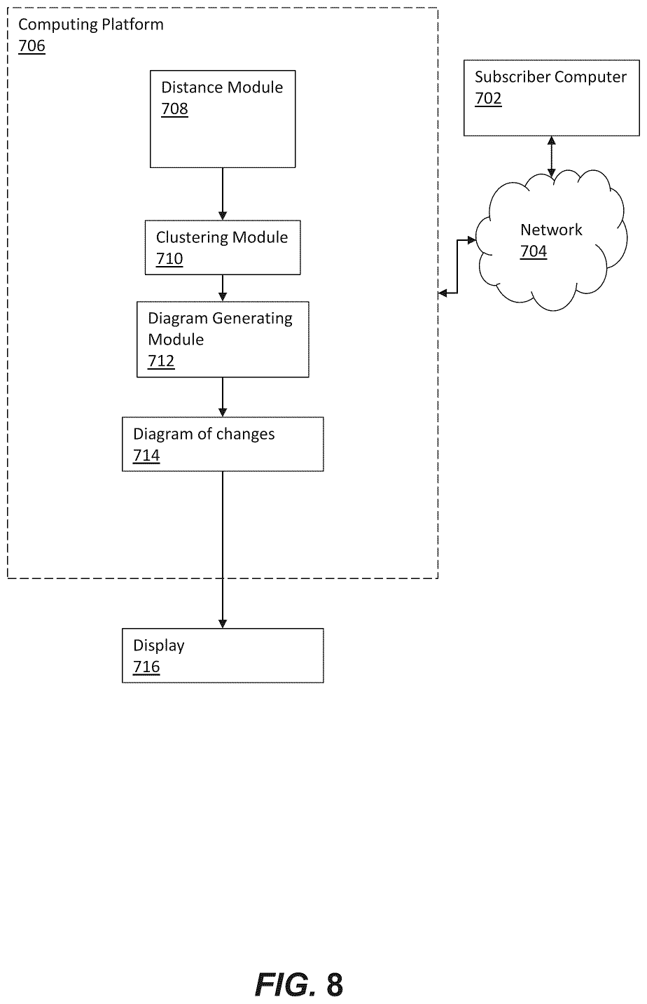

FIG. 8 shows an embodiment of a system for implementing a diagram generating protocol. A subscriber computer 702 is connected through a network 704 to a computing platform 706. The computing platform can be configured as a diagram generative platform that can analyze compounds in three-dimensional spaces, which can be used to generate a diagram or an alchemical network. The subscriber computer uploads subscriber data to the computing platform 706. For example, the subscriber data can include a set of compounds each having an initial condition and a final condition. A distance module 708 may be configured to determine comparative distances of areas of each of the plurality of chemical compounds. In some embodiments, the distance module may analyze the compounds in three-dimensional to determine the distances. For example, physical configurations and electrical potentials of each compound in the set of compounds may be used to determine the distances. The distance module 708 may be configured to calculate the distances in both the initial condition and the final condition. For example, the distances between different regions of within the compound may be calculated with respect to the compound in the first state and in the second state. In some embodiments, the distances may be determined using a machine learning algorithm. For example, the distance module 708 may include automated machine learning algorithms to determine the distances.

A clustering module 710 may be configured to cluster the compounds based on the calculated distances. For example, the clustering module 710 may form one or more clusters in which the clusters group together the compounds based on similarities and/or dissimilarities, represented using the calculated distances. In some embodiments, the clustering module 710 can implement different clustering algorithms to form the one or more clusters. For example, the clustering module 710 may implement k-means clustering, density-based clustering, hierarchical clustering, among others.

A diagram generating module 712 can be configured to generate a diagram of changes 714. The diagram generating module 712 can obtain the clusters generated using the clustering module 710 and generate the diagram 714 based on the clusters. For example, the diagram generating module 712 may generate an alchemical graph including nodes and edges, in which nodes present compounds, and the edges represent transitions from one compound to another compound. In some embodiments, the diagram generating module 712 can limit the number of edges included in the diagram 714. For example, the diagram generating module 712 can limit the number of edges to be equal to or less than N(N−1)/2, in which N represents the number of compounds represented using the graph. More preferably, the number of the edges may be equal to N*ln(N).

In some embodiments, the computing platform 706 may be connected to a display 716. For example, the computing platform 706 may be communicatively coupled to the display 716 over a wired and/or wireless connection. In some embodiments, the display 716 can be a part of the subscriber computer 702. In some embodiments, the computing platform 706 can communicate the diagram of changes 714 to the display 716. The display 716 can be configured to display the diagram 714. A user or a subscriber providing the set of compounds to the computing platform 706 can view and analyze the diagram 714 via the display 716.

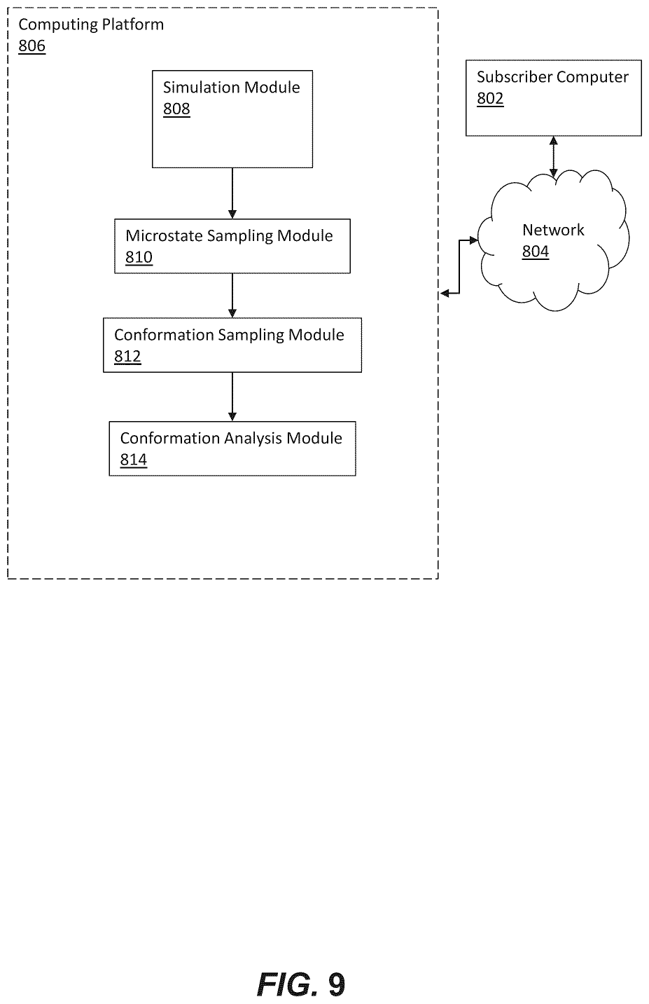

FIG. 9 shows an embodiment of a computing platform for implementing an analysis of an alchemical transformation. In some embodiments, a computing platform 806 can be configured to implement the analysis. In some embodiments, the computing platform 806 may be a part of the computing platform 706 of FIG. 8. In other embodiments, the computing platform 806 can be a separate platform from the computing platform 706 of FIG. 8. A subscriber computer 802 is connected through a network 804 to the computing platform 806. In some embodiments, the subscriber computer 802 may be the same computer as the subscriber computer 702 of FIG. 8. In some embodiments, a set of compounds including at least a first compound and a second compound may be provided to the computing platform 806 via the subscriber computer 802. For example, a simulation module 808 may obtain the set of compounds from the subscriber computer 802. In these and other embodiments, the simulation module 808 may perform simulation for the first compound. For example, the simulation module may predict how the first compound may interact with a specific target. In some embodiments, the simulation process may be described with more detail with respect to block 502 of FIG. 6 of the present disclosure. For example, the simulation module 808 may correspond to the computational model described with respect to FIG. 6. In some embodiments, the simulation module 808 may generate a trajectory that represents the movement of the first compound and the specific protein over time.

A microstate sampling module 810 can be configured to sample microstates for the alchemical transformation process between the first compound and the second compound. In some embodiments, the microstates may represent intermediate states during the transformation process from the first compound to the second compound. For example, a computational simulation may be run using the first compound and the second compound to predict the transformation process. In some embodiments, multiple simulation trajectories may be determined between the first compound and the second compound. For example, the first compound could transform to the second compound via different pathways. In some embodiments, the microstate sampling module 810 can be configured to determine at least one simulation trajectory including one or more microstates.

A conformation sampling module 812 can be configured sample different conformations of the second compound with respect to the target. For example, the second compound may have different conformations in interacting with the target. The conformation sampling module 812 can be configured to sample the conformations from relevant phase space. In some embodiments, the sampling may be done from equilibrium similar to the first compound. In some embodiments, a conformation analysis module 814 may analyze the conformations of the second compound generated using the conformation sampling module 812. For example, the conformation analysis module 814 can be configured to estimate binding abilities of different conformations relative to the target. The conformation analysis module can be further configured to verify the estimation of the binding ability through a partition function ratio, which may be described in further detail with respect to FIG. 6 of the present disclosure.

In some embodiments, a computer-implemented method can include: obtaining, by a diagram generating platform, a plurality of chemical compounds each having an initial condition and a final condition; computing, by a distance module, comparative distances of areas of each of the plurality of chemical compounds in the initial condition and in the final condition, wherein the distances are calculated based on a physical configuration and electrical potential of each chemical compound; forming, by a clustering module, one or more clusters of the plurality of chemical compounds based on the computed comparative distances of the areas of each of the plurality of chemical compounds; and generating, by a diagram generating module, a diagram of changes for the plurality of chemical compounds, the diagram including a plurality of nodes and a plurality of edges, wherein each node indicates one chemical compound, and each edge is a transition from one chemical compound to another chemical compound of the plurality of chemical compounds.

In some embodiments, a computer-implemented method can include: obtaining a plurality of compounds including at least a first compound and a second compound, wherein each compound has an ability to bind to a target; determining, by a simulation module, an equilibrium simulation for the first compound of the plurality of compounds for binding to the target; sampling, by a microstate sampling module, microstates for a transformation process between the first compound and the second compound; determining, by the microstate sampling module, at least one simulation trajectory of the transformation process; and sampling, by a conformation sampling module, conformations of the second compound from the at least one simulation trajectory and the target.

In some embodiments, one or more non-transitory computer readable media are provided that store instructions that in response to being executed by one or more processors, cause a computer system to perform operations, the operations comprising performing the computer methods described herein for generating a diagram of changes.

In some embodiments, a computer system can include: one or more processors; and one or more non-transitory computer readable media storing instructions that in response to being executed by the one or more processors, cause the computer system to perform operations, the operations comprising performing the methods described herein for generating a diagram of changes or perturbation graph.

In some embodiments, a method for analyzing changes in binding free energy for a plurality of chemical compounds in different conditions can include providing the plurality of chemical compounds each having an initial condition and a final condition; computing comparative distances of areas of each of the plurality of chemical compounds in the initial condition and in the final condition, which is based on a physical configuration and electrical potential of each chemical compound; forming groupings of the plurality of chemical compounds based on the computed comparative distances of the areas of each of the plurality of chemical compounds; and generating a diagram of changes for the plurality of chemical compounds, wherein a point indicates one chemical compound and a connection is a transition from one chemical compound to another chemical compound of the plurality of chemical compounds. In some embodiments, the method can further include visually displaying the generated diagram of changes. In some embodiments, a quantity of connections can be equal to or less than a certain mathematical expression involving a variable. In some embodiments, the plurality of chemical compounds can be provided in a digital environment. In some embodiments, the comparative distances are computed using a machine learning algorithm. In some embodiments, the groupings of the plurality of chemical compounds are formed using a clustering algorithm. In some embodiments, the point indicating one chemical compound is represented by a unique identifier. In some embodiments, connection representing a transition from one chemical compound to another is represented by a unique identifier. In some embodiments, the initial condition and the final condition of each chemical compound are represented by unique identifiers. In some embodiments, the physical configuration and the electrical potential of each chemical compound are represented by unique identifiers. In some embodiments, the comparative distances of the areas of each of the plurality of chemical compounds are represented by unique identifiers. In some embodiments, the groupings of the plurality of chemical compounds are represented by unique identifiers. In some embodiments, the diagram of changes for the plurality of chemical compounds is represented by a unique identifier. In some embodiments, the quantity of connections can be equal to or less than a certain mathematical expression involving a variable, and the variable is represented by a unique identifier.

In some embodiments, a system for analyzing changes in binding free energy for a plurality of chemical compounds in different conditions can include: a processor configured to provide the plurality of chemical compounds each having an initial condition and a final condition, compute comparative distances of areas of each of the plurality of chemical compounds in the initial condition and in the final condition, which is based on a physical configuration and electrical potential of each chemical compound, form groupings of the plurality of chemical compounds based on the computed comparative distances of the areas of each of the plurality of chemical compounds, and generate a diagram of changes for the plurality of chemical compounds, wherein a point indicates one chemical compound and a connection is a transition from one chemical compound to another chemical compound of the plurality of chemical compounds. In some embodiments, the system can further include a display configured to visually display the generated diagram of changes. In some embodiments, the processor is further configured to determine that a quantity of connections can be equal to or less than a certain mathematical expression involving a variable. In some embodiments, the processor is further configured to provide the plurality of chemical compounds in a digital environment. In some embodiments, the processor is further configured to compute the comparative distances using a machine learning algorithm, form the groupings of the plurality of chemical compounds using a clustering algorithm, and generate the diagram of changes using a graph theory algorithm.

In some embodiments, a computer-implemented method for performing energy computation can include: receiving data related to one or more molecular interaction processes; utilizing one or more energy computation methods to compute free energy differences associated with said molecular interaction processes; modifying a molecular identity of a part of a computational system in one or more intermediate states; altering an interaction potential in said intermediate states; engaging with a molecular environment in said intermediate states; altering, adding, or removing one or more atomic entities in said intermediate states. In some embodiments, the method can further include containing essential parts for energy computation methods in a computational workflow; creating a network design method in said computational workflow; depicting one or more state representations in said computational workflow; utilizing a dynamics computation method in said computational workflow; calculating an energy estimation method in said computational workflow; utilizing one or more computational techniques in said computational workflow. In some embodiments, the method can further include utilizing spatial features of a plurality of chemical entities by a network generation tool to generate one or more transformation networks; contrasting a spatial shape and an electrostatic property in a plurality of chemical compounds; evaluating a molecular transformation for said chemical compounds. In some embodiments, the method can further include executing a structural transformation type; including said structural transformation type in a chemical research practice; restricting approaches for constructing transformation graphs by a network building approach and a similarity measure; generating network connections in a molecular network produced by a network generation tool approx. equals N*ln(N); and maintaining an accuracy measure by a number of said network connections. In some embodiments, said molecular interaction processes are associated with binding of a molecular entity to a molecular binding site, transfer of a molecular entity from an initial phase to a final phase, effects of protein modifications on molecular interaction strengths or stability measures. In some embodiments, said intermediate states are non-physical. In some embodiments, said transformation networks are alchemical networks. In some embodiments, said spatial shape and said electrostatic property are 3D features of said chemical entities. In some embodiments, said structural transformation type is scaffold hopping or ring opening/closing. In some embodiments, said chemical research practice is medicinal chemistry practice. In some embodiments, said network building approach is MCS or structural similarity.

In some embodiments, a method for estimating the binding ability of chemical entities to a biological target can include performing one or more simulation processes for a first chemical entity; performing one or more simulation processes for a second chemical entity; collecting microstates for a transformation process between the first and second chemical entities; collecting conformational states of the second chemical entity and the biological target interaction through one or more simulation trajectories; leading to a reliable estimation of the binding ability through relevant conformational states; collecting microstates of the second chemical entity through one or more independent simulation processes in a complex with a biological entity; ensuring a reliable estimation of the binding ability through a ratio function for bound entities; and indicating the bound or unbound state of entities through a state indicator function. In some embodiments, the first and second chemical entities are ligands. In some embodiments, the biological target is a protein. In some embodiments, the simulation processes for the first and second chemical entities are equilibrium simulations. In some embodiments, the transformation process between the first and second chemical entities is an alchemical transformation. In some embodiments, the simulation trajectories of the second chemical entity and the biological target interaction are sampled from the relevant phase space. In some embodiments, the relevant conformational states are sampled from the simulation trajectory of the second chemical entity and the biological target complex. In some embodiments, the independent simulation processes in a complex with the biological entity are equilibrium simulations. In some embodiments, the ratio function for bound entities is a partition function ratio.

In some embodiments, a method for calculating changes in binding free energy for a substance in different state can include providing a plurality of substances, wherein each substance has an ability to bind to a target substance; determining an equilibrium simulation for a first substance of the plurality of substances for binding a target substance; determining an equilibrium simulation for a second substance of the plurality of substances for binding the target substance; analyzing a transformation from the first substance to the second substance; sampling microstates of the transformation; and providing at least one transformation having the sampled microstates. In some embodiments, the method can further include sampling conformations of the second substance from a simulate trajectory of complex of the second substance and the target substance. In some embodiments, the method can further include determining a relevant phase space of conformations of the first substance and/or the second substance. In some embodiments, the method can further include sampling conformations of substance B in a gas phase space. In some embodiments, the method can further include sampling different conformations of substance B. In some embodiments, the method can further include sampling microstates of the second substance from a relevant phase space. In some embodiments, the method can further include performing an independent equilibrium simulation of the second substance complexed with the target substance as a protein. In some embodiments, the method can further include estimating a partition function ratio for the first substance and second substance bound with the target substance. In some embodiments, the substance is a molecule, such as a small molecule, macromolecule, polypeptide, protein, antibody, oligonucleotide, nucleic acid (e.g., RNA, DNA, etc.), polypeptide, carbohydrate, lipid, or combinations thereof, whether natural or synthetic.

In some embodiments, the molecules that are generated are analyzed, and one or more specific molecules that fit specified condition criteria are selected. The selected one or more molecules are then selected and synthesized before being tested with one or more cells to determine whether or not the synthesized molecules actually satisfy the condition.

Once one or more molecules are generated, the model can categorize the molecules according to whatever profile is desirable. A specific physical property, such as certain chemical moieties or 3D structure can be prioritized, and then a molecule with a profile that matches the desired profile is selected and synthesized. As such, an object selector (e.g., molecule selector), which can be a software module, selects at least one molecule for synthesis, which can be done by filtering as described herein. The selected molecule is then provided to an object synthesizer, where the selected object (e.g., selected molecule) is then synthesized. The synthesized object (e.g., molecule) is then provided to the object validator (e.g., molecule validator, which tests the object to see if it satisfies the condition or property, or to see if it is biologically active for a specific use. For example, a synthesized object that is a molecule can be tested with live cell cultures or other validation techniques in order to validate that the synthesized molecule satisfies the desired property.

Once a generated object is selected, then the method includes validating the selected object. The validation can be performed as described herein. When the object is a molecule, the validation can include synthesis and then testing with live cells.

In some embodiments, a method can include selecting a selected substance that corresponds with the selected generated data or that corresponds with the desired properties; and validating the selected substance. In some embodiments, the method may include: obtaining a physical version for the selected substance; and testing the physical version to have a desired property or biological activity. Also, in any method the obtaining of the physical version of the substance can include at least one of synthesizing, purchasing, extracting, refining, deriving, or otherwise obtaining the physical substance. The physical substance may be a molecule or other. The methods may include the testing involving assaying the physical substance in a cell culture. The methods may also include assaying the physical substance by genotyping, transcriptome-typing, 3-D mapping, ligand-receptor docking, before and after alchemical transformations, reference thermodynamic state analysis, target thermodynamic analysis, or combinations thereof. Preparing the physical version for the selected generated substance can often include synthesis when the physical substance is a new molecular entity. Accordingly, the methods may include selecting a generated object that is not part of the original dataset or previously known.

In some embodiments, the method can include: the obtaining of the physical form of the selected compound includes at least one of synthesizing, purchasing, extracting, refining, deriving, or otherwise obtaining the physical object; and/or the testing includes assaying the physical form of the selected object in a cell culture; and/or assaying the physical form of the selected compound by genotyping, transcriptome-typing, 3-D mapping, ligand-receptor docking, before and after alchemical transformations, reference thermodynamic state analysis, target thermodynamic state analysis, or combinations thereof.

Artificial Neural Networks (ANNs) are computing systems inspired by the biological neural networks that constitute animal brains. An ANN is based on a collection of connected units or nodes called artificial neurons, which loosely model the neurons in a biological brain. Each connection, like the synapses in a biological brain, can transmit a signal to other neurons. An artificial neuron receives a signal then processes it and can signal neurons connected to it. The “signal” at a connection is a real number, and the output of each neuron is computed by some non-linear function of the sum of its inputs. The connections are called edges. Neurons and edges typically have a weight that adjusts as learning proceeds. The weight increases or decreases the strength of the signal at a connection. Neurons may have a threshold such that a signal is sent only when the aggregate signal crosses that threshold. Typically, neurons are aggregated into layers. Different layers may perform different transformations on their inputs. Signals travel from the first layer (the input layer) to the last layer (the output layer), possibly after traversing the layers multiple times.

Deep Neural Networks (DNNs) are ANNs with one or more hidden layers. These networks, due to their complex structure and a large number of trainable parameters, make it possible to solve problems more efficiently. Autoencoders are a subset of DNNs that learn the hidden representation of objects. Objects can be different mathematically formalized objects, for example—strings, graphs, or pictures. An autoencoder includes two parts—an encoder and a decoder. An encoder is an encoding function that maps an object to a point (e.g., latent point) in a numerical space with a specified dimension. This numerical space is called latent space. A decoder is a decoding function that maps a point in latent space to an object in the object space. For training, these networks use reconstruction loss, a function that penalizes the model for differences between the input (encoder input) and output (decoder output) representations of an object.

Generative models (GM) are a subclass of DNNs that enable the generation of objects. Unlike standard DNNs that predict the properties of objects, these networks are trained in such a way as to generate new objects in the future without input data. These models learn the distribution of objects (e.g., distributional learning) and then try to generate samples from this distribution.

Autoencoder-based generative models (ABGM) are generative models that are based on autoencoder architecture. For the generating process, these models use different mechanics for learning and interacting with the latent space. The most popular representatives of this class of models are Adversarial Autoencoder (AAE) and Variational Autoencoder (VAE). Both of these networks use different learning techniques, the goal of which is to ensure that the distribution of representations of objects in the latent space is as close as possible to some given distribution, such as normal (normal distribution). If the network is trained well, then the generation process will be to randomly sample points from this given distribution and decode them using a decoder part of the model. Another type of generative model is the Generative Adversarial Network (GAN), which is a network that uses a latent space for sampling molecules, but it is not an autoencoder-based generative model since it does not have an encoder part of the network. This model uses the mechanism of an adversarial game for learning latent space distribution.

Distributional learning generative models generate random molecules by default. However, sometimes one wants to generate objects that satisfy given properties. This formulation of the problem is called conditional generation.

A Recurrent Neural Network (RNN) is a type of neural network that contains loops, which allows information to be stored within the network. RNN uses their reasoning from previous experiences to inform the upcoming events. Recurrent models are usually used for tasks related to the textual representation of input data, such as, for example, SMILES representation of molecules. The Long Short Term Memory Network (LSTM) is an advanced RNN, which is a sequential network that allows information to persist. It is capable of handling the vanishing gradient problem that can be faced by an RNN.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

Cross-reference is made to the following incorporated references: U.S. Pat. Nos. 11,568,961; 11,403,521; US 2015/0178442; US 2020/0090049; US 2020/0082916; US 2020/0258594; US 2022/0310196; US 2021/0233621; US 2021/0271980; US 2021/0287067; US 2021/0383898; US 2022/0172802; US 2022/0406404; EP 3289501; WO 2021/165887; and WO 2021/229454.

1. A computer-implemented method, comprising:
obtaining, by a diagram generating platform, a plurality of chemical compounds each having a reference state and a target state;
computing, by a distance module, comparative distances of areas of each of the plurality of chemical compounds in the reference state and in the target state, wherein the distances are calculated based on a physical state and electrical potential of each chemical compound;
forming, by a clustering module, one or more clusters of the plurality of chemical compounds based on the computed comparative distances of the areas of each of the plurality of chemical compounds; and
generating, by a diagram generating module, a diagram of changes for the plurality of chemical compounds, the diagram including a plurality of nodes and a plurality of edges, wherein each node indicates one chemical compound, and each edge is an alchemical transformation from one chemical compound to another chemical compound of the plurality of chemical compounds.

2. The method of claim 1, further comprising visually displaying the generated diagram of changes.

3. The method of claim 1, wherein a number of the edges is equal to or less than N*(N−1)/2, N representing a number of the chemical compounds.

4. The method of claim 1, wherein a number of the edges of the diagram is equal to or less than N*ln(N), N representing a number of the chemical compounds.

5. The method of claim 1, wherein the edges are weighted.

6. The method of claim 1, wherein the diagram is an alchemical network.

7. The method of claim 1, wherein the plurality of chemical compounds is provided in a digital environment.

8. The method of claim 1, wherein the comparative distances are determined using a machine learning algorithm.

9. The method of claim 1, wherein the one or more clusters are determined using a clustering algorithm.

10. The method of claim 1, wherein the nodes are represented using unique identifiers.

11. The method of claim 1, wherein the nodes are represented using structural properties.

12. A computer-implemented method comprising:
obtaining a plurality of compounds including at least a first compound and a second compound, wherein each compound has an ability to bind to a target or has been hypothesized to bind a target;
determining, by a simulation module, an equilibrium simulation for the first and the second compounds of the plurality of compounds for binding to the target;
sampling, by a conformation sampling module, transition microstates for an alchemical transformation between the microstates of first compound and the second compound; and
determining, by the conformation sampling module, at least one simulation trajectory of the alchemical transformation.

13. The method of claim 12, further comprising:
estimating binding abilities based on the conformations;
verifying the estimation of the binding abilities through a partition function ratio; and
indicating bound or unbound state of the compounds.

14. The method of claim 12, wherein the conformations are sampled in equilibrium.

15. The method of claim 12, wherein the conformations are sampled in a relevant phase space.

16. The method of claim 15, wherein the relevant phase space is determined based on molecular mechanics, quantum mechanics or any combination of methods.

17. The method of claim 12, wherein specific definitions of the lambda functions proportional to the arcsine and smoothstep functions used to schedule the mixing of nonbonded interactions to the Hamiltonian of simulated system of interest.

18. The method of claim 12, wherein the equilibrium simulation is performed using molecular dynamics simulation, Monte Carlo simulation, or quantum mechanics simulation.

19. The method of claim 12, wherein the target is a protein.

20. The method of claim 12, wherein the plurality of compounds are drugs.

21. The method of claim 12, wherein the plurality of compounds is selected based on abilities to interact with another molecular entity, such as a protein target, an enzyme target, or a receptor target.

22. One or more non-transitory computer readable media storing instructions that in response to being executed by one or more processors, cause a computer system to perform operations, the operations comprising performing the method of claim 1.

23. A computer system comprising:
one or more processors; and
one or more non-transitory computer readable media storing instructions that in response to being executed by the one or more processors, cause the computer system to perform operations, the operations comprising performing the method of claim 1.
